(19) United States
(12) Patent Application Publication
(10) Pub. No.: US 2005/0124829 A1
(43) Pub. Date: Jun. 9, 2005

(54) Brominated esters, methods for their preparation and use thereof as flame retardants

(76) Inventor: Dov Beruben, Beer-Sheva (IL)

Correspondence Address: Kevin D. McCarthy;Roach Brown McCarthy & Gruber, 1620 Liberty Building, Buffalo, NY, 14202, US

(21) Appl. No.: 10/504,621

(22) PCT Filed: Feb. 17, 2003
(86) PCT No.: PCT/IL03/00123

(30) Foreign Application Priority Data
Feb. 21, 2002 (IL) 148326

(51) Int. Cl. C09K 21/00; C07C 69/52; C07C 67/02

(52) U.S. Cl. 560/254; 560/193; 252/609

(57) ABSTRACT

Polyhalogenated benzyl esters having general formula (1), wherein X is halogen; n is an integer from 1 to 5; Y is an aliphatic chain, such as having at least 4 carbon atoms or a radical (2) wherein X has the same meaning as above, p is an integer from 0 to 5 and Z is (CH2)m where m is zero or an integer greater than zero. Y may be a saturated aliphatic chain, a linear or branched aliphatic chain having one or more unsaturations, or a substituted or unsubstituted aliphatic chain.

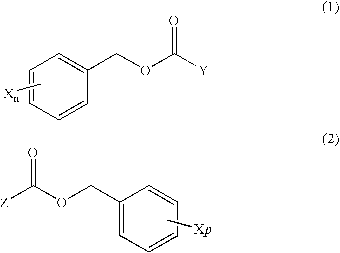

### FIELD OF THE INVENTION

This invention relates to new chemical compounds which are polyhalogenated benzyl esters, to a process for their preparation, to their use as flame retardants (FR), particularly in polymeric systems, and to compositions of matter comprising a polymeric matrix and said esters.

### BACKGROUND OF THE INVENTION

Flame retardants, particularly comprising bromine, are well known in the art and widely used. U.S. Pat. No. 5,072,028 discloses the preparation of bromo-substituted aromatic esters of α,β-unsaturated acids of the formula 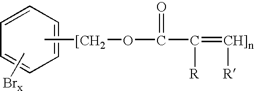
wherein n is 1 or 2, x=5 and R and R″ are hydrogen or alkyl Such compounds are prepared by reacting a salt of α,β-unsaturated acid and an alkali with a bromo-substituted benzyl halide in an inert substantially water-immiscible solvent and in the presence of a phase-transfer catalyst.

This invention proposes new polyhalogenated benzyl esters, other than those the preparation of which is described in the aforesaid patent. The purposes of this invention are to provide a wide range of flame retardants with different physical and chemical properties, and which permit, by the choice of the most suitable ones in each individual case, to prepare fire retarded polymer compositions having optimal properties.

### SUMMARY OF THE INVENTION

The novel polyhalogenated benzyl esters of this invention have the general formula: 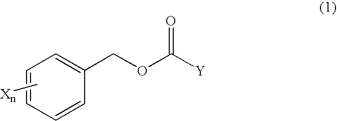
wherein X is halogen and n is an integer from 1 to 5; Y is an aliphatic chain having at least 4 carbon atoms or a radical 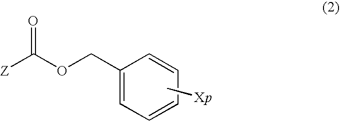
wherein X has the same meaning as above, p is an integer from 0 to 5, and Z is (CH2)m wherein m is zero or an integer greater than zero.

When Y is an aliphatic chain, it may be saturated or unsaturated with one or more unsaturations and may be linear or branched. It may be unsubstituted or substituted, e.g. have a functional group such as an alcohol, amine or thiol group.

When Y is the radical (2), the general formula of compounds of the invention is: 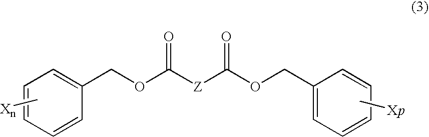
wherein n is an integer from 1 to 5, p is an integer from 0 to 5 and Z may be saturated or unsaturated with one or more unsaturations, may be linear or branched, and may be unsubstituted or substituted, e.g. may have a functional group such as alcohol, amine or thiol group.

The compounds of the invention are generally white, odorless solids and should be preferably in powder form to be used as fire retardants, as hereinafter described.

A particularly useful group of the above compounds is that in which n=5 and X is bromine, viz. they are compounds pentabrominated in the aromatic ring.

Another aspect of the invention is a method for the preparation of the aforesaid new polyhalogenated benzyl esters, which comprises reacting a polyhalogenated benzyl halide and the appropriate mono- or polycarboxylic acid or lactone in the presence of a base, solvents, and optionally a phase transfer catalyst (PTC). The reaction is preferably a one-pot synthesis in which the following preferential conditions apply:
the base is a metal hydroxide, preferably NaOH or KOH;
the phase transfer catalyst may be any known one, e.g. a quaternary ammonium salt, for example tetrabutyl ammonium bromide;
the temperature is from 40° C. to 140° C. or more, more preferably between 80° C. and 110° C.;
the reaction time is in the range of 2 to 8 hours, more preferably about 6 hours;
the solvent can be water, organic solvents, a mixture of water and water-miscible organic solvents, or a mixture of water and water-immiscible solvents.

The reaction can also be carried out in water without organic solvent, but this is not desirable because it requires a longer reaction time. Examples of water-miscible solvents are monosubstituted ethylene glycols, such as 2-ethoxyethanol or 2-methoxyethanol, or disubstituted ethylene glycol as 1,2-dimethoxyethane or 1,2-diethoxyethane. The reaction can also be carried out for example in toluene, tetrahydrofuran, cyclohexane, methylene chloride, acetonitrile, alcohols, etc., and in the carboxylic acid itself as solvent when it's a liquid.

The carboxylate prepared from the addition of the base to the aqueous solution of the carboxylic acid or lactone (these latter preferably in aqueous solution) may be added to the polyhalogenated benzyl halide instead of separately adding the carboxylic acid or lactone and the base.

This may be desirable if the end product is easily hydrolyzed under basic conditions.

At the end of the reaction, the product obtained is separated by filtration or by extraction with organic solvent and evaporation. Other purification steps may be carried out: for instance, if there are traces of carboxylic acid in the final material, this latter can be poured into water and the carboxylic acid be neutralized by adding a base, in an amount which, together with the amount originally introduced, is equivalent to the carboxylic acid used, and then filtering the mixture. The removal of salts, such as NaBr or KBr, formed in the reaction may also be effected as needed.

Another aspect of the invention is the use of the polyhalogenated benzyl esters of the invention as flame retardants, particularly in polymeric systems. They are used in the conventional way in which other flame retardants are used. Generally they are obtained from their preparation as powders, and as such, can be compounded with the polymer in any suitable way. The amount of the polyhalogenated benzyl ester used as flame retardant may vary widely according to the particular ester used and the particular polymer to which it is added, but a most common range is from 5 to 25 wt % of the polymer.

Another aspect of the invention are compositions of matter comprising a polymer matrix and one or more flame retardants of formula (1) or (3). The polymer matrix may comprise a thermoplastic or a thermosetting polymer.

Particular examples of such polymers are high impact polystyrene (HIPS), polypropylene, poly(butylterephtalate) and polyurethanes, but the invention is not limited to them and comprises compositions based on any thermoplastic or thermosetting polymers.

### DETAILED DESCRIPTION OF PREFERRED EMBODIMENTS

The preparation of some polyhalogenated benzyl esters according to the invention will now be described for purposes of illustration and not of limitation.

### EXAMPLE 1

### General Procedure for the Preparation of Carboxylic Acid Pentabromobenzyl Ester

Into a one-liter four-necked flask equipped with a mechanical stirrer, thermometer, condenser and a dropping funnel were charged 566 g of pentabromobenzylbromide (1 mole), 105.6 g of isobutyric acid (1.2 mole), 300 ml of 2-ethoxyethanol and 200 ml of water. The flask and the contents were heated to 105° C. and 88 g of an aqueous solution of NaOH (50% weight in water, 1.1 mole) was added dropwise.

The reaction could be monitored by HPLC or GC, following the consumption of the PBB-Br. The mixture was stirred for 6 hours at this temperature and after completion of the reaction, 200 ml of water was added.

The contents were cooled to room temperature while a precipitation of the product occurred. After filtration of the solvent, the solid was poured into 500 ml of water and an additional 8 g of aqueous solution of NaOH (50% in water, 0.1 mole) were added. After stirring, the product was filtered out and dried to constant weight.

A white solid (m.p. 157.3-157.4° C.) was obtained. The weight of the product was 566.7 g, corresponding to a 99% yield based on PBB-Br. % Br: Calcd-69.76%; found-69%.

### EXAMPLE 2

Following the procedure outlined in Example 1, but using 172.8 g of 2-ethyl hexanoic acid (1.2 mole) instead of isobutyric acid, the corresponding pentabrominated ester was obtained in 99% yield (624 g), based on PBB-Br. M.P. 63.7-64.8° C. % Br: Calcd-63.54%; found-62.82%.

### EXAMPLE 3

Following the procedure outlined in Example 1, but using 172.8 g of octanoic acid (1.2 mole) instead of isobutyric acid, the corresponding pentabrominated ester was obtained in 94.6% yield (595 g), based on PBB-Br. M.P. 68.1-69.2° C.

### EXAMPLE 4

Following the procedure outlined in Example 1, but using 206.4 g of decanoic acid (1.2 mole) instead of isobutyric acid, the corresponding pentabrominated ester was obtained in 98% yield (643 g), based on PBB-Br. % Br: Calcd-60.82%; found-60.16%.

### EXAMPLE 5

Following the procedure outlined in Example 1, but using 240 g of lauric acid (1.2 mole) instead of isobutyric acid, the corresponding pentabrominated ester was obtained in 96% yield (657.5 g), based on PBB-Br. % HPLC: 98%.

### EXAMPLE 6

Following the procedure outlined in Example 1, but using 273.6 g of myristic acid (1.2 mole) instead of isobutyric acid, the corresponding pentabrominated ester was obtained in 95% yield (677 g), based on PBB-Br. % HPLC: 93%.

### EXAMPLE 7

Following the procedure outlined in Example 1, but using 307.2 g of palmitic acid (1.2 mole) instead of isobutyric acid, the corresponding pentabrominated ester was obtained in 99% yield (773.6 g), based on PBB-Br. % HPLC: 80%.

### EXAMPLE 8

Following the procedure outlined in Example 1, but using 340.8 g of stearic acid (1.2 mole) instead of isobutyric acid, the corresponding pentabrominated ester was obtained in 97.7% yield (751 g), based on PBB-Br. % HPLC: 85%.

### EXAMPLE 9

### Preparation of Succinic Acid Dipentabromobenzyl Ester

Into a one-liter four-necked flask equipped with a mechanical stirrer, thermometer, condenser and a dropping funnel were charged 679.2 g of pentabromobenzylbromide (1.2 mole), 82.6 g of succinic acid (0.7 mole), 300 ml of 2-ethoxyethanol and 200 ml of water. The flask and the contents were heated to 105° C. and 104 g of an aqueous solution of NaOH (50% weight in water, 1.3 mole) was added dropwise. The mixture was stirred for 6 hours at this temperature and after completion of the reaction, 200 ml of water was added.

The contents were cooled to room temperature while a precipitation of the product occurred. After filtration of the solvent, the solid was poured into 500 ml of water and an additional 8 g of aqueous solution of NaOH (50% in water, 0.1 mole) were added. After stirring, the product was filtered out and dried to constant weight.

A white solid was obtained. The weight of the product was 581 g, corresponding to an 89% yield based on PBB-Br. MP>280° C.

### EXAMPLE 10

### Preparation of Hexanedioic Acid Dipentabromobenzyl Ester

Following the procedure outlined in Example 9, but using 102.2 g of adipic acid (0.7 mole) instead of succinic acid, the corresponding pentabrominated ester was obtained in 96% yield (642 g), based on PBB-Br. KP. 192° C. % Br. Calcd-71.64%; found-70.66%.

### EXAMPLE 11

### Preparation of octadec-9-enoic Acid Pentabromobenzyl Ester

Following the procedure outlined in Example 1, but using 376 g of oleic acid (90% purity, 1.2 mole) instead of isobutyric acid, the corresponding pentabrominated ester was obtained after extraction with methylene chloride in 78% yield (603 g) based on PBB-Br. The product is an oil at ambient temperature, that could be solidified at sub-ambient temperature.

### EXAMPLE 12

### Preparation of 2-hydroxy-propionic Acid Pentabromobenzyl Ester

Following the procedure outlined in Example 1, but using 127.06 g of lactic acid (85% in water, 1.2 mole) instead of isobutyric acid, the corresponding pentabrominated ester was obtained in 87% yield (500 g), based on PBB-Br. M.P. 167-169° C.

### EXAMPLE 13

### Preparation of 6-hydroxy-hexanoic Acid Pentabromobenzyl Ester

Into a one-liter four-necked flask equipped with a mechanical stirrer, thermometer, condenser and a dropping funnel were charged 566 g of pentabromobenzylbromide (1 mole) and 300 ml of 2-ethoxyethanol. The flask and the contents were heated to 105° C. and an aqueous solution of c-caprolactone (136.8 g, 1.2 mole) and 88 g of a solution of NaOH (50% weight in water, 1.1 mole) into 200 ml of water was added dropwise. The mixture was stirred for 6 hours at this temperature and after completion of the reaction, 200 ml of water was added.

The contents were cooled to room temperature while a precipitation of the product occurred. After filtration of the solvent, a white solid was obtained. The weight of the product was 575 g, corresponding to a 96% yield based on PBB-Br. M.P.: 99.7-101.3° C.

### EXAMPLE 14

### Preparation of 2-ethylhexanoic Acid Pentabromobenzyl Ester in Water for Comparison with Example 2

Following the procedure outlined in example 1 but using 14.15 g of PBBBr (0.025 mole), 4.32 g of 2-ethylhexanoic acid (0.03 mole), 2.2 g of an aqueous solution of NaOH (0.0275 mole, 50% in water) into 50 ml of water (instead of the mixture water/2-ethoxyethanol used in Example 2) for 30 hours. The HPLC chromatogram revealed the formation of 50% of the expected product.

### EXAMPLE 15

### Preparation of 2-ethylhexanoic Acid Pentabromobenzyl Ester in Water/Toluene

Following the procedure outlined in example 14 but replacing the water by 50 ml of a mixture of water/toluene (1/l) and tetrabutylammonium bromide (0.8 gr., 1 mole %) as phase transfer catalyst for 6 hours. The HPLC chromatogram revealed 85% of the expected product.

### EXAMPLE 16

### Preparation of 2-ethylhexanoic Acid Pentabromobenzyl Ester Using 2-ethylhexanoic Acid as Solvent

Following the procedure outlined in example 14 but using 18 g of 2-ethylhexanoic acid (0.125 mole) without water, for 30 h. The HPLC chromatogram revealed 70% of the expected product.

### EXAMPLE 17

### Preparation of 2-ethylhexanoic Acid Pentabromobenzyl Ester Using 1,2-dimethoxyethane as Solvent

Following the procedure outlined in example 14 using 50 ml of a solution of water/1,2-dimethoxymethane (1/l) as solvent, for 6 h. The HPLC chromatogram revealed 97% of the expected product.

### EXAMPLE 18

### Preparation of 2-ethylhexanoic Acid Pentabromobenzyl Ester Using THF as Solvent

Following the procedure outlined in example 14 using 50 ml of THF instead of water for 6 h. The HPLC chromatogram revealed 95% of the expected product.

### EXAMPLE 19

### Preparation of 2-ethylhexanoic Acid Pentabromobenzyl Ester Using Cyclohexane as Solvent

Following the procedure outlined in example 14 using 50 ml of cyclohexane instead of water for 30 h. The HPLC chromatogram revealed 10% of the expected product.

### EXAMPLE 20

### Preparation of 2-ethylhexanoic Acid Pentabromobenzyl Ester in Water/Acetonitrile

Following the procedure outlined in example 14 replacing the water by 50 ml of a mixture of water/acetonitrile (1/l) for 30 h. The HPLC chromatogram revealed 70% of the expected product.

Any carboxylic acid having at least one acid group, or any lactone are suitable for a reaction with a polyhalogenated benzyl halide benzyl. The following are examples of compounds that may be used, though others can also be used: pentanoic acid, heptanoic acid, nonanoic acid, undecanoic acid, tridecanoic acid, pentadecanoic acid, heptadecanoic acid, nonadecanoic acid, eicosanoic acid, cyclobutane carboxylic acid, 2-methyl butyric acid, 1-methyl cyclopropane carboxylic acid, 2-methyl cyclopropane carboxylic acid, cyclopentane carboxylic acid, cyclohexane carboxylic acid, propiolactone, γ-butyrolactone, β-butyrolactone, α-methyl-γ-butyrolactone, γ-valerolactone, λ-valerolactone, γ-caprolactone, γ-octanoic lactone, γ-nonanoic lactone, γ-decanolactone, λ-decanolactone, undecanoic γ-lactone, undecanoic λ-lactone, dodecanolactone, ω-pentadecanolactone, myristoleic acid, palmitoleic acid, 11-eicosanoic acid, linoleic acid, linolenic acid, 11,14-eicosadienoic acid, 8,11,14-eicosatrienoic acid, arachidonic acid, 5,8,11,14,17-eicosapentaenoic acid, oxalic acid, malonic acid, glutaric acid, pimelic acid, subaric acid, azeloic acid, sebacic acid, undecanedioic acid, 1,1-undecanedicarboxylic acid, dodecanedioic acid, 1,12-dodecanedicarboxylic acid, hexadecanedioic acid, fumaric acid, traumatic acid, 1,3-adamantane dicarboxylic acid.

The following examples illustrate the use of the polyhalogenated benzyl esters of the invention as flame retardants for polymers. The polymers to which the examples refer are high-impact polystyrene (HIPS) and polyester (PBT), but in the same way, the esters could be used as flame retardants for other polymers.

### EXAMPLE 21

### Application of Pentabromobenzyl Esters as Fire Retardants in Polystyrene

The products of Examples 1, 2, 4 and 10 were incorporated into high-impact polystyrene (HIPS), containing MB-AO (a master batch made of 80 wt % of Sb2O3 and 20 wt % of a carrier polymer) and Irganox B-225 (blend of Irganox 1010—tetrakis[methylene(3,5-di-tert-butyl-4-hydroxyhydrocinnamate)]methane—and Irgafos 168-Tris(2,4-di-tertbutylphenyl)phosphite) as antioxidant following standard conditions and testing in the EL-94 burning test.

The results are summarized in the following tables.

TABLE I
HIPS formulation passing UL-94 V-2 classification test

| | | Exam- | Exam- | Exam- | Exam- |
|---|---|---|---|---|---|
| Components | units | ple 1 | ple 2 | ple 4 | ple 10 |
| HIPS | % | 86.1 | 74.6 | 84.7 | 86.4 |
| FR | % | 10.2 | 19.1 | 11.6 | 9.9 |
| MB-AO A0112 | % | 3.5 | 6.1 | 3.5 | 3.5 |
| Irganox B-225 | % | 0.2 | 0.2 | 0.2 | 0.2 |
| Bromine | % | 7 | 12 | 7 | 7 |
| Br/Sb atomic ratio | | 4.5 | 4.5 | 4.5 | 4.5 |
| Rating UL-94 for 1.6 mm | | V-2 | V-2 | V-2 | V-2 |

TABLE II
HIPS formulation passing UL-94 V-0 classification test

| Components | units | Example 1 | Example 4 | Example 10 |
|---|---|---|---|---|
| HIPS | % | 76.3 | 73.8 | 76.7 |
| FR | % | 17.4 | 19.9 | 17 |
| MB-AO A0112 | % | 6.1 | 6.1 | 6.1 |
| Irganox B-225 | % | 0.2 | 0.2 | 0.2 |
| Bromine | % | 12 | 12 | 12 |
| Br/Sb atomic ratio | | 4.5 | 4.5 | 4.5 |
| Rating UL-94 for 1.6 mm | | V-0 | V-0 | V-0 |

### EXAMPLE 22

### Application of Pentabromobenzyl Esters as Fire Retardants in PBT

The product of example 10 was incorporated into Poly (Butylene Terephtalate), containing MB.A.O (a master batch made of 80 wt % of Sb₂O₃ and 20% of a carrier polymer), and Irganox 1010-Tetrakis[methylene(3,5-di-tert-butyl-4-hydroxyhydrocinnamate)] methane as antioxidant, and PTFE (polytetrafluoroethylene) Hostaflon TF 2071 as antidripping, following standard conditions and testing in the UL-94 burning test.

The results are summarized in the following table.

TABLE III
Composition and test results

| Components | units | Example 10 |
|---|---|---|
| PBT Celanex 2500 | % | 80.8 |
| product | % | 11.3 |
| MB-AO M0112 | % | 6.1 |
| Irganox 1010 | | 0.2 |
| PFTE Hostaflon TF2072 | % | 0.2 |
| Bromine | % | 8 |
| Rating UL-94 for 0.8 mm | | V-0 |
| Rating UL-94 for 1.6 mm | | V-0 |

### EXAMPLE 23

### Application of Pentabromobenzyl Esters as Fire Retardants in Polyurethane

The product of examples 2 and 12 were incorporated into standard flexible polyurethane foam used as polymeric matrix. The polyol (100 parts polyol per one hundred parts resin (phr)), water (4.2 phr), surfactant (1.1 phr), Phosflex 31L ex Akzo Nobel (2.5-2.8 phr), flame retardant and catalysts were weighed and mixed using a high speed mixer at about 3000 rpm. The polyol used in this example was a polyether with terminal hydroxyl groups. The molecular weight was about 4800 and the hydroxyl number was 46 mg KOH/g. TDI (54.8 phr) was added under the hood, mixed for 10 sec including the pouring time (the timer was started when TDI was added). The TDI is an 80:20 mixture of 2,4- and 2,6-toluene-diisocyanate. The mixture was then quickly poured into a shoebox with dimensions 33×20×20 cm³. The usual cream time (from the moment TDI was added till the foam started to rise) was about 15 sec. The usual blow off time (measured from the moment TDI was added till the foam stopped rising and CO₂ was suddenly released through the upper surface) was about 130-140 sec. The resulting bun was let to cool under the hood for 24 hours, dismantled from the shoebox and cut into 30×7.5×1.27 cm³ specimens. At least 10 specimens were tested, 5 specimens from each formulations after conditioning at room temperature and 50±5% relative humidity for 24 hours, and another set of 5 specimens from each formulation after aging for 24 hours at 104° C. The specimens were subjected to the vertical burning test in a special cabinet as required by the California TB 117, Section A, Part I. The flammability was tested by bottom ignition for a 12-second interval using a vertical burner butane flame with a length of 40 mm. For a formulation to pass the test, it is required that the maximum char length would be below 20 cm, the average char length below 15 cm, the average after-flame time below 5 sec, the maximum after-time time below 10 sec, and the maximum after-glow time below 15 sec. These requirements should be met for both sets of specimens. All the formulations containing the novel pentabromobenzylbenzyl esters of present invention passed the California TB 117, Section A, Part I test, indicating that they are able to provide a high level of fire retardancy efficiency to flexible polyurethane foams.

TABLE IV

| | | | | Cal 117 | Cal 117 |
|---|---|---|---|---|---|
| Flame | Ester | Phosflex 31L | Bromine | RT | 104° C. |
| retardant | phr | phr | Wt % | conditioning | aging |
| Ester of | 8.4 | 2.8 | 3.1 | pass | pass |
| Example 2 | | | | | |
| Ester of | 7.5 | 2.5 | 3.1 | pass | pass |
| Example 12 | | | | | |

While a number of examples have been given by way of illustration, it should be understood that the invention can be carried out with many modifications, variations and adaptations, without departing from its spirit or exceeding the scope of the claims.

1. Polyhalogenated benzyl esters having the general formula: 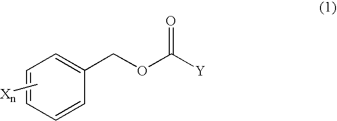
wherein X is halogen; n is an integer from 1 to 5; Y is an aliphatic chain having at least 4 carbon atoms or a radical 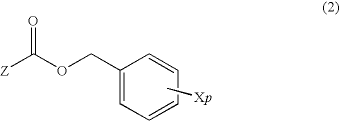
wherein X has the same meaning as above, p is an integer from 0 to 5 and Z is (CH2)m where m is zero or an integer greater than zero.

2. Polyhalogenated benzyl esters according to claim 1, wherein Y is a saturated aliphatic chain.

3. Polyhalogenated benzyl esters according to claim 1, wherein Y is a linear or branched aliphatic chain having one or more unsaturations.

4. Polyhalogenated benzyl esters according to claim 1, wherein Y is a substituted or unsubstituted aliphatic chain.

5. Polyhalogenated benzyl esters according to claim 1, having the general formula 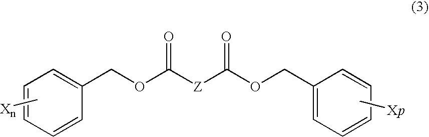
wherein X is halogen; n is an integer from 1 to 5; p is an integer from 002 to 5; and Z is (CH2)m where m is zero or an integer greater than zero and is saturated or unsaturated, linear or branched, unsubstituted or substituted.

6. Polyhalogenated benzyl esters according to claim 1, wherein n=5 and X is bromine.

7. Method for the preparation of the polyhalogenated benzyl esters having the general formula: 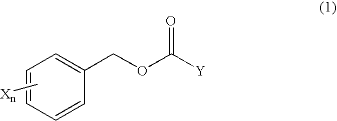
wherein X is halogen: n is an integer from 1 to 5; Y is an aliphatic chain having at least 4 carbon atoms or a radical 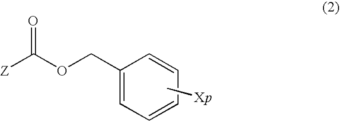
wherein X has the same meaning as above, p is an integer from 0 to 5 and Z is (CH2)m where m is zero or an integer greater than zero, which comprises
reacting a polyhalogenated benzyl halide and the appropriate mono- or polycarboxylic acid or lactone in the presence of a base, a solvent, and optionally a phase transfer catalyst.

8. Method according to claim 7, wherein the base is a metal hydroxide.

9. Method according to claim 7, wherein the base is NaOH or KOH.

10. Method according to claim 7, wherein the phase transfer catalyst is a quaternary ammonium salt.

11. Method according to claim 7, wherein the reaction is carried out at temperatures from 40° C. to 140° C.

12. Method according to claim 11, wherein the reaction is carried out at temperatures from 80° C. and 110° C.

13. Method according to claim 11, wherein the reaction time is in the range of 2 to 8 hours.

14. Method according to claim 13, wherein the reaction time is about 6 hours

15. Method according to claim 7, wherein the solvent is selected from the group consisting of water, organic solvents, mixtures of water and water-miscible organic solvents, or mixtures of water and water-immiscible organic solvents.

16. Method according to claim 7, wherein the base and the carboxylic acid are combined as carboxylate.

17. Polyhalogenated benzyl esters according to claims 1 are used as fire retardants.

18. Polyhalogenated benzyl esters according to claims 1 are used as fire retardants of thermoplastic and thermosetting polymeric systems.

19. Polyhalogenated benzyl esters according to claim 18, wherein the polymeric system is a polymeric foam.

20. Polyhalogenated benzyl esters Use according to claim 19, wherein the polymeric foam is a polyurethane foam.

21. Polyhalogenated benzyl esters according to claim 17, wherein the polyhalogenated benzyl esters are used in amounts range from 5 to 25 wt % of the polymer.

22. Polyhalogenated benzyl esters according to claim 1 wherein the polyhalogenated benzyl ester is a flame retardant composition in a polymer matrix.

23. The composition according to claim 22, wherein the polymer matrix comprises a thermoplastic or a thermosetting polymer.

24. The composition according to claim 22, wherein the polymer is selected from the group consisting of high impact polystyrene (HIPS), polypropylene, poly(butyl terephtalate), and polyurethanes.
